(19) United States
(12) Patent Application Publication
(10) Pub. No.: US 2025/0359801 A1
(43) Pub. Date: Nov. 27, 2025

(54) LEAD SET WIRE MANAGEMENT

(71) Applicant: KPR U.S., LLC, Mansfield, MA (US)

(72) Inventors: David SELVITELLI, Suffield, CT (US); Liang DAYANG, Dongguan City (CN); Stephane JEAN-PIERRE, Rocky Hill, CT (US); Mo XIBAO, Dongguan (CN)

(21) Appl. No.: 19/219,473

(22) Filed: May 27, 2025

Related U.S. Application Data
(60) Provisional application No. 63/651,863, filed on May 24, 2024.

(51) Int. Cl. A61B 5/30; A61B 5/273; A61B 5/308

(52) U.S. Cl. CPC A61B 5/303; A61B 5/273; A61B 5/308; A61B 2562/222

(57) ABSTRACT

A cable management clip includes a first engagement surface for supporting the cable set, a cover movably attached to a base and defining an engagement surface for contacting the plurality of cables of the cable set. The cover is movable between an open position permitting the plurality of cables of the cable set to be received in a space between the base and the cover, and a closed position where the cover at least partially closes the space between the base and the cover. The clip may further include a locking tab attached to the cover and defining another engagement surface for contacting the plurality of cables of the cable set, the locking tab being movable to a closed position fixing the cable management clip in place along the plurality of cables of the cable set.

### CROSS REFERENCE TO RELATED APPLICATION

This application claims priority to U.S. Provisional Application No. 63/651,863, titled: LEAD SET WIRE MANAGEMENT, the entirety of which is incorporated by reference herein.

### FIELD

The present disclosure generally relates to wire management, and in particular, to wire management for an electrode lead set.

### SUMMARY

A summary of certain embodiments disclosed herein is set forth below. It should be noted that these aspects are presented merely to provide the reader with a summary of these certain embodiments and that these aspects are not intended to limit the scope of this disclosure. This summary is not an extensive overview of all contemplated aspects and is intended to neither identify key or critical elements of all aspects nor delineate the scope of any or all aspects. Its purpose is to present some concepts of one or more aspects in a simplified form as a prelude to the more detailed description below.

Some aspects of this disclosure include a cable management clip for use with a cable set including a plurality of cables, the cable management clip including: a base defining a first engagement surface for supporting the cable set; a cover movably attached to the base and defining a second engagement surface for contacting the plurality of cables of the cable set, the cover being movable between an open position permitting the plurality of cables of the cable set to be received in a space between the base and the cover, and a closed position where the cover at least partially closes the space between the base and the cover; and a locking tab movably attached to the cover and defining a third engagement surface for contacting the plurality of cables of the cable set, the locking tab being movable between an open position permitting the cable management clip to be moveably constrained along the plurality of cables of the cable set, and a closed position fixing the cable management clip in place along the plurality of cables of the cable set.

Some aspects of this disclosure combinable with any of the above mentioned aspects include a cable management clip, wherein the cover is pivotally attached to the base by a first living hinge, and the locking tab is pivotally attached to the cover by a second living hinge.

Some aspects of this disclosure combinable with any of the above mentioned aspects include a cable management clip, wherein the first living hinge has a pivot axis that extends perpendicularly to a pivot axis of the second living hinge.

Some aspects of this disclosure combinable with any of the above mentioned aspects include a cable management clip, wherein the base, cover, and locking tab are formed as one piece of material.

Some aspects of this disclosure combinable with any of the above mentioned aspects include a cable management clip, wherein the first engagement surface includes a first portion and a second portion, the first portion being configured to engage at least one of the plurality of cables of a first size, and the second portion being configured to engage at least one of the plurality of cables of a second size larger than the first size.

Some aspects of this disclosure combinable with any of the above mentioned aspects include a cable management clip, wherein the second engagement surface includes a first portion and a second portion, the first portion of the second engagement surface being configured to engage cables of a first size, and the second portion of the second engagement surface being configured to engage cables of a second size larger than the first size.

Some aspects of this disclosure combinable with any of the above mentioned aspects include a cable management clip, wherein the third engagement surface includes a first portion and a second portion, the first portion of the third engagement surface being configured to engage cables of a first size, and the second portion of the third engagement surface being configured to engage cables of a second size larger than the first size.

Some aspects of this disclosure combinable with any of the above mentioned aspects include a cable management clip, wherein the locking tab includes a locking bar on the third engagement surface, the locking bar being configured to apply a friction force to the plurality of cables of the cable set to fix the cable management clip in place along the plurality of cables when the locking tab is moved to the closed position.

Some aspects of this disclosure combinable with any of the above mentioned aspects include a cable management clip wherein the base includes a first securement element and the cover include a second securement element that engages the first securement element when the cover is moved to the closed position to hold the cover in the closed position.

Some aspects of this disclosure combinable with any of the above mentioned aspects include a cable management clip, wherein the locking tab includes a third securement element that engages the first securement element when the locking tab is moved to the closed position to hold the locking tab in the closed position.

Some aspects of this disclosure combinable with any of the above mentioned aspects include a cable management clip, wherein the locking tab includes a fourth securement element and the base includes a fifth securement element, the fourth securement element engaging the fifth securement element when the locking tab is moved to the closed position to hold the locking tab in the closed position.

Some aspects of this disclosure combinable with any of the above mentioned aspects include a cable management clip, wherein the cable set includes first cables, the cable management clip further including a cable retainer disposed on the base and configured for releasably retaining a second cable of the cable set to the cable management clip when the locking tab is in the closed position.

Some aspects of this disclosure combinable with any of the above mentioned aspects include a cable management clip, wherein the cable retainer includes a first cable retainer, the cable management clip further including a second cable retainer disposed on the base and configured for releasably retaining a third cable of the cable set to the clip when the locking tab is in the closed position.

Some aspects of this disclosure combinable with any of the above mentioned aspects include a cable management clip in combination with the cable set, which may be an electrode leadset.

Aspects of the disclosure include the description of a cable management clip for use with a lead set including: a base defining a first engagement surface for supporting first cables of the lead set; a cover movably attached to the base and defining a second engagement surface for contacting the first cables of the lead set, the cover being movable between an open position permitting the first cables of the lead set to be received in a space between the base and the cover, and a closed position where the cover at least partially closes the space between the base and the cover; and a cable retainer disposed on the base and configured for releasably retaining a second cable of the lead set to the cable management clip.

Some aspects of this disclosure combinable with any of the above mentioned aspects include a cable management clip, wherein the base includes a height extending between a top edge and a bottom edge of the base, and a width extending between opposite sides of the base, the first cables of the lead set extending along the height of the base, and a cable extension plane extending across the width of the base, the cable retainer being disposed on one of the sides of the base.

Some aspects of this disclosure combinable with any of the above mentioned aspects include a cable management clip, wherein the base and cover are configured to retain one of the first cables of the lead set along a first cable axis, and the cable retainer is configured to retain the second cable along a second cable axis, the first cable axis and second cable axis being disposed in the cable extension plane.

Some aspects of this disclosure combinable with any of the above mentioned aspects include a cable management clip, the cable retainer includes a first cable retainer, the cable management clip further including a second cable retainer disposed on the base and configured for releasably retaining a third cable to the cable management clip when the cover is in the closed position, the second cable retainer being disposed on the other of the sides of the base.

Some aspects of this disclosure combinable with any of the above mentioned aspects include a cable management clip, wherein the second cable retainer is configured to retain the third cable along a third cable axis, the first cable axis and third cable axis being disposed in the cable extension plane.

Some aspects of this disclosure combinable with any of the above mentioned aspects include a cable management clip, further including a locking tab movably attached to the cover and defining a third engagement surface for contacting the first cables of the lead set, the locking tab being movable to fix the cable management clip in place along the first cables, the cable retainer releasably retaining the second cable of the lead set to the cable management clip when the cable management clip is fixed in place along the first cables.

### BACKGROUND

When a patient requires monitoring for observation, treatment, or a combination of both, such as in a medical environment, e.g., a hospital, nursing home, or assisted living facility, the patient's vital signs and other health indicators may be monitored in order to continually and accurately assess the patient's well-being. One such vital sign is the monitoring of the heart via an electrocardiogram, which may be commonly referred to as an EKG and/or ECG.

Electrocardiograph (ECG) monitors are widely used to obtain medical (i.e. biopotential) signals containing information indicative of the electrical activity associated with the heart and pulmonary system. To obtain medical signals, ECG electrodes are applied to the skin of a patient in various locations. The electrodes, after being positioned on the patient, connect to an ECG monitor by a set of ECG lead wires. The distal end of the ECG lead wire, or portion closest to the patient, may include a connector which is adapted to operably connect to the electrode to receive medical signals from the body. The proximal end of the ECG lead set is operably coupled to the ECG monitor and supplies the medical signals received from the body to the ECG monitor.

To monitor events of the heart via an ECG, a series of 3, 5, 6, 10, or 14 or more electrodes may be placed on a patient to sense electrical signals corresponding to activity of a patient's heart. For example, each of the electrodes may be used to allow the charge carriers (electrons) within the electrodes to communicate with the charge carriers (ions) within the body via electrochemical exchange. ECG electrodes on the body surface of a patient allows for voltage changes within the body to be recorded and/or displayed to a heath professional after adequate amplification of the signal.

### BRIEF DESCRIPTION OF THE DRAWINGS

FIG. 1 is a schematic illustration of a patient with a plurality of electrode assemblies attached to the patient and a lead set connected to the electrode assemblies;

FIG. 2 is an illustration of a lead set assembly of the present disclosure;

FIG. 3 is a perspective view of a connector of the lead set assembly;

FIG. 4 is a top view of the connector;

FIG. 5 is a side view of the connector;

FIG. 6 is an enlarged partial perspective view of the lead set assembly showing a wire management clip in an open position;

FIG. 7 is an enlarged partial perspective view of the lead set assembly showing the wire management clip in a closed and unlocked position;

FIG. 8A is an enlarged partial top view of the lead set assembly showing the wire management clip in a closed and locked position;

FIG. 8B is an enlarged partial top view of an alternative lead set assembly showing the wire management clip in a closed and locked position;

FIG. 9 is a perspective view of the wire management clip;

FIG. 10 is another perspective view of the wire management clip;

FIG. 11 is a top end view of the wire management clip;

FIG. 12 is a bottom end view of the wire management clip; and

FIG. 13 is a top view of the wire management clip.

Corresponding reference characters indicate corresponding parts throughout the drawings.

### DETAILED DESCRIPTION

### 1. Explanation/Examples of Terms

In the description herein, the following definitions of selected terms employed herein are provided. The definitions include various examples and/or forms of components that fall within the scope of a term and that may be used for implementation. The examples are not intended to be limiting. Further, it will be obvious to one skilled in the art that the present disclosure may be practiced without these specific details. In other instances, well-known methods, procedures, and components have not been described in detail so as to not unnecessarily obscure aspects of the present disclosure.

Throughout the disclosure, the term “substantially” or “approximately” may be used as a modifier for a geometric relationship between elements or for the shape of an element or component. While the terms substantially or approximately are not limited to a specific variation and may cover any variation that is understood by one of ordinary skill in the art to be an acceptable variation, some examples are provided as follows. In one example, the terms substantially or approximately may include a variation of less than 10% of the dimension of the object or component. In another example, the terms substantially or approximately may include a variation of less than 5% of the object or component. If the terms substantially or approximately are used to define the angular relationship of one element to another element, one non-limiting example of the terms may include a variation of 5 degrees or less. These examples are not intended to be limiting and may be increased or decreased based on the understanding of acceptable limits to one of ordinary skill in the art.

For purposes of the disclosure, directional terms are expressed generally with relation to a standard frame of reference when the devices described herein is installed and in an in-use orientation.

In this disclosure, terms such as “a,” “an,” and “the” are not intended to refer to only a singular entity, but include the general class of which a specific example may be used for illustration. The terms a, an, and the, are used interchangeably with the term at least one. The phrases at least one of and comprises at least one of followed by a list refers to any one of the items in the list and any combination of two or more items in the list. All numerical ranges are inclusive of their endpoints and non-integral values between the endpoints unless otherwise stated.

The terms “first,” “second,” “third,” and “fourth” are used in this disclosure. It will be understood that, unless otherwise noted, those terms are used in their relative sense only. In particular, in some aspects certain components may be present in interchangeable and/or identical multiples (e.g., pairs). For these components, the designation of first, second, third, and/or fourth may be applied to the components merely as a matter of convenience in the description of one or more of the aspects of the disclosure.

The terms “disposed on,” “disposed along,” “disposed with,” or “disposed toward” and variations thereof may define one element can be integral with another element, or that one element can be a separate structure bonded to or placed with or placed near another element.

The term “at,” for example when referring to something being located at a specific location, is intended to include any one or more of: proximate, on, near, adjacent to or within the specific location.

In the description, certain terms may be used such as “forward,” “front,” “rear,” “back,” “up,” “down,” “upper,” “lower,” “horizontal,” “vertical,” “left,” “right,” “longitudinal,” “lateral,” and the like. These terms are used, where applicable, to provide some clarity of description when dealing with relative relationships. However, these terms are not intended to imply absolute relationships, positions, and/or orientations. For example, with respect to an object, an upper surface can become a lower surface by turning the object over.

The term “proximal” is defined with respect to an object, element, or user. For example, the term proximal may refer to the part or portion closer to the user.

The terms “comprising,” “including,” and “having” are intended to be inclusive and mean that there may be additional elements other than the listed elements. Additionally, references to “one aspect” or “an aspect” of the present disclosure are not intended to be interpreted as excluding the existence of additional features or components, either described in this application or that would have been understood by one of ordinary skill in the art after reading this disclosure, that also incorporate the recited features.

### II. Detailed Examples

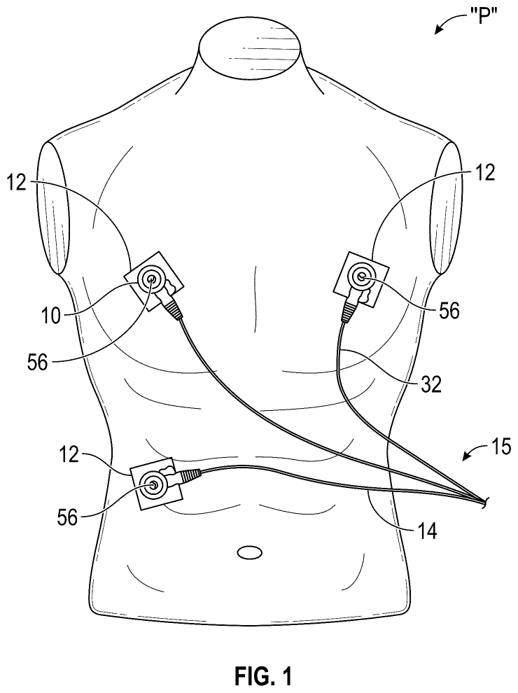

One or more aspects of the present disclosure pertain to lead sets for biomedical electrodes that may be attached to a patient's skin in order to measure (or monitor) the electrical activity of the patient's heart and produce a signal that is delivered to a monitoring device, in addition to methods of use thereof. Referring to FIG. 1, a plurality of electrode assemblies 12 may be placed at various locations on a patient's body to monitor electrical activity of the heart and electrode connectors 10 may be connected to the electrodes. Movement by the patient can cause lead cables 32 extending from the connectors to become tangled. However, by providing the wire management features as described herein, the lead cables 32 can be held together preventing the wires from becoming tangled. The wire management features also provide for a low profile solution to organizing the lead cables 32.

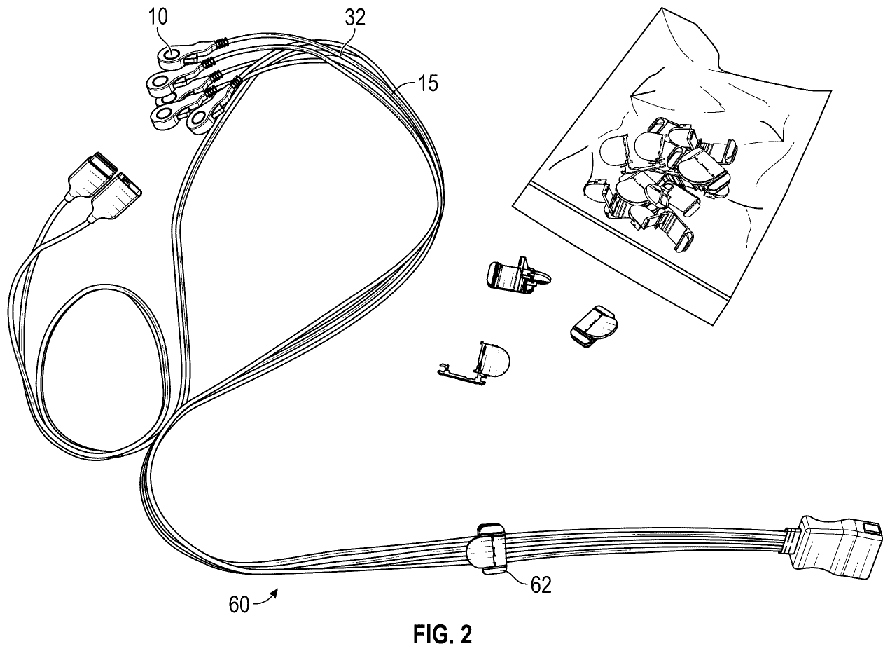

Referring to FIGS. 1 and 2 generally, each electrode assembly 12 (e.g., an ECG electrode assembly) is configured to be secured to the patient's skin at one side of the electrode and connect to a dedicated connector 10 at an opposite side of the electrode. The connectors 10 may be part of a lead set 15, including the cables 32, and that is connectable to a monitoring device (not shown) so that the electrodes can measure (or monitor) the electrical activity of the heart and produce a signal that is delivered to the monitoring device for observation by a clinician. Although the present invention has particular application in the management of electrical wires, it could be used in other embodiments to manage conduits carrying fluid among other things.

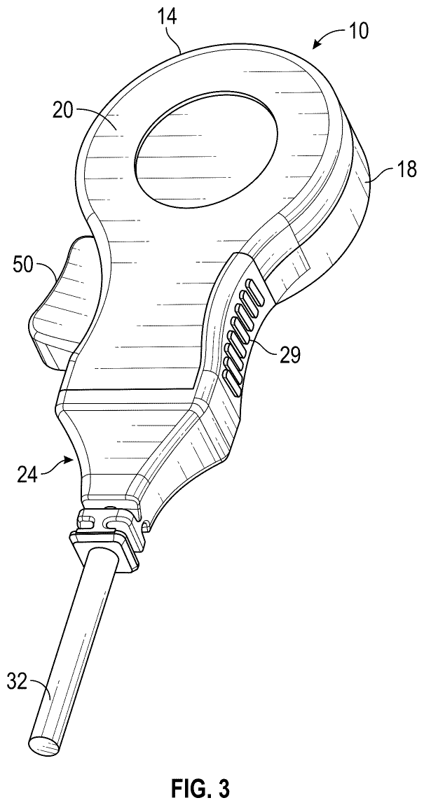

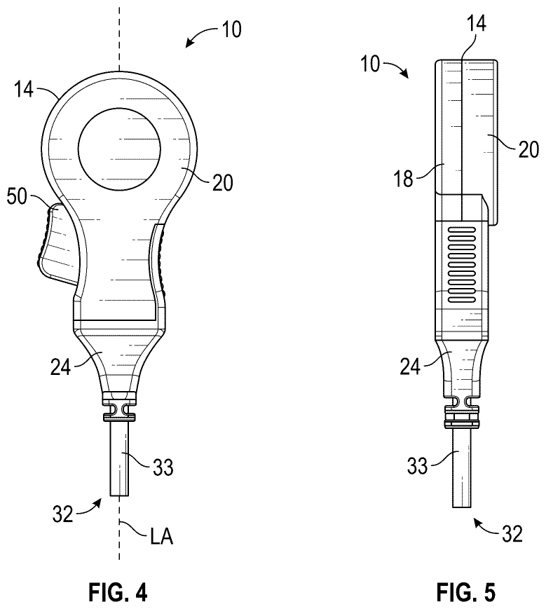

Referring to FIGS. 3-5 generally, each patient connector 10 is configured for connection to an electrode 12 and/or a connection component for use therewith. Each connector 10 may include a housing 14 defining an internal cavity (not shown). The housing 14 includes a lower member 18 and an upper member 20 whereby the internal cavity is formed between the lower and upper members. The housing 14 may be formed from a non-conducting material including, without limitation, thermoplastics and/or elastomeric polymer such as polybutylene terephthalate (PBT), polyethylene terephthalate (PET), polyvinyl chloride (PVC), acrylonitrile butadiene styrene (ABS), thermoplastic polyurethanes (TPU), thermoplastic vulcanates (TPV), polypropylene (PP), polyethylene (PE), and/or fiber-reinforced polymer (FRP). In one embodiment, the housing 14 may comprise an injection molded polymer that electrically insulates the patient from the conductive components disposed in the housing. In the illustrated embodiment, the lower and upper members 18, 20 are separate components attached together by suitable means such as by adhesive, ultrasonic welding, or heat welding. The lower housing member 18 defines an opening (not shown) configured to receive a stud 56 (FIG. 1) of the electrode 12. However, the housing 14 could have other configurations without departing from the scope of the disclosure. For example, the housing 14 could be formed by a single housing component. In other examples of the disclosure, the individual components and features described herein (e.g., the hinges and portions connected via the hinges) may be separate components that are connected to one another via any one or combination of fasteners, interlocking features, adhesive(s), ultrasonic welding, and/or heat welding.

A strain relief 24 may be attached to the housing 14 and extend generally along (e.g., be centered on) a longitudinal axis LA of the housing. The strain relief 24 may be formed from a flexible material. In one embodiment, the strain relief 24 is formed from a low durometer PVC. Still other materials for the strain relief 24 are envisioned without departing from the scope of the disclosure. A contact member (not shown) may also be supported in the housing 14 and electrically connected to a lead wire 32 that is electrically connected to the monitoring device (e.g., ECG monitor). The lead wire 32 may include an external portion (i.e. shielded wire 33 extending away from the connector) and an internal portion (i.e., portion of the lead wire disposed inside the connector). The lead wire 32 may be supported at its exit location from the housing 14 by the strain relief 24. A lever 50 may be pivotably connected to the housing 14. In use, lever 50 is actuatable to allow the stud 56 of the electrode 12 to be received through the opening in the housing 14 to attach the connector 10 to the electrode.

Referring to FIG. 1, the lead set 15 may be adapted for connection to conventional biomedical electrode assemblies 12 (e.g., an ECG electrode assembly). A typical ECG electrode assembly may include an electrically conductive layer and a backing layer (broadly, a base). The electrode assembly 12 has a patient contact side and a connector side. The contact side of the electrode may include biocompatible conductive gel or adhesive for affixing the electrode to a patient's body for facilitating an appropriate electrical connection between a patient's body and the electrode assembly. The connector side of the electrode may incorporate a metallic press stud 56 extending in transverse relation to the base having a bulbous profile whereby an upper portion has a greater cross-sectional dimension than a lower portion for coupling the electrode to the connector 10. In use, the clinician removes a protective covering from the contact side to expose the gel or adhesive, affixes the electrode to the patient's body, and attaches the appropriate ECG lead wire connector (e.g., connector 10) to the press stud 56. The electrode assembly may have other constructions as known by those of ordinary skill in the art.

It is noted that while electrode assemblies are provided as examples (in this case, a lever-style electrode assembly), aspects of this disclosure are applicable management of any one or combination of different type/styles of cables, wires, and/or tubes in the medical context or otherwise (e.g., snap electrodes or multi-parameter cables).

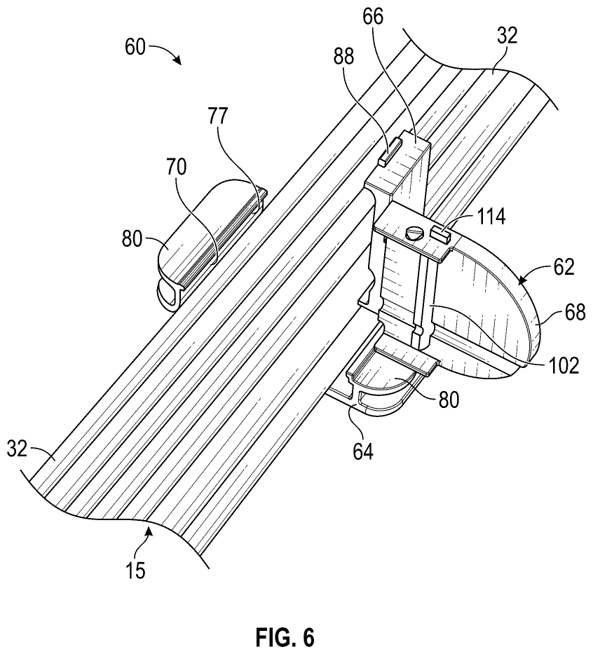

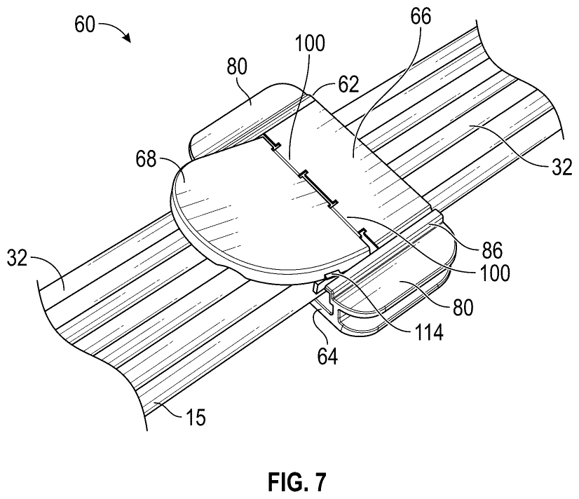

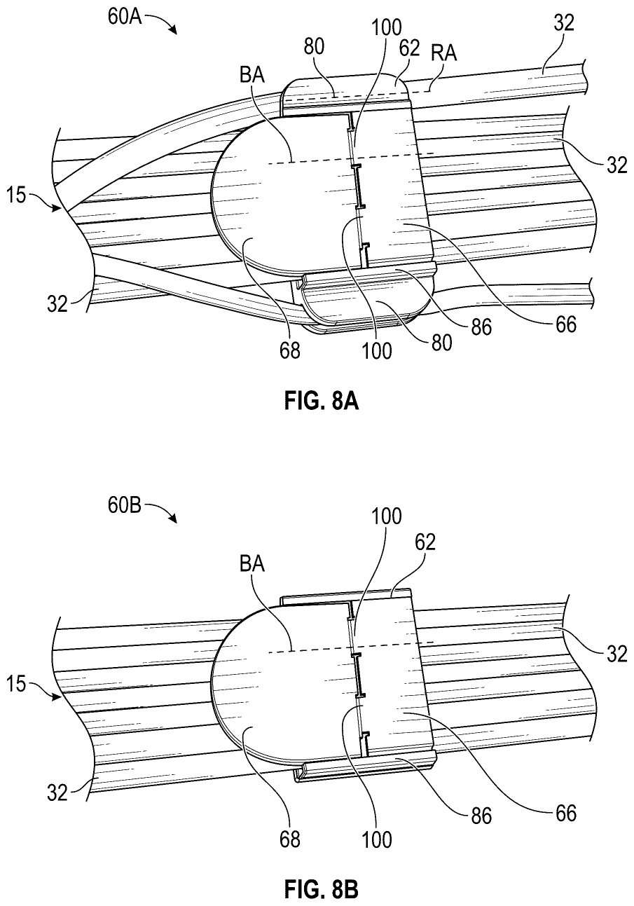

Referring to FIGS. 2 and 6-8, a lead set assembly of the present disclosure is generally indicated at 60 and includes the lead set 15 and a lead set management clip 62 (may be hereinafter referred to as “clip” for brevity). The clip 62 is configured to receive and retain the cables 32 of the lead set 15 to organize the cables. In the illustrated embodiment, a single clip 62 is shown on the lead set 15. However, multiple clips 62 may be provided on the lead set 15. In one embodiment the clip 62 comprises a unitary body including a base portion 64 (broadly, a base), a first top portion 66 (broadly, a cover) pivotally attached to the base portion, and a second top portion 68 (may be interchangeably referred to as a “locking tab” herein) pivotally attached to the first top portion. In the illustrated example, the clip 62 is formed as a single piece of material. The first top portion 66 is pivotable between an open position (FIG. 6) wherein the first top portion 66 is pivoted away from the base portion 64, and a closed position (FIG. 7) wherein the first top portion is pivoted toward the base portion and into engagement with the base portion to secure the first top portion to the base portion. When the first top portion 66 is pivoted to the open position, the cables 32 of the lead set 15 may be installed or otherwise received into a space 70 between the base portion 64 and the first and second top portions 66, 68, and in the closed position (e.g., as shown in FIG. 7), the base portion 64 and first top portion 66 are configured to retain the clip 62 to the cables 32 or otherwise constrain the clip 62 with respect to the cables 62. However, in the closed position, clip 62 is slideably constrained on the cables 32 or otherwise remains unlocked such that the clip 62 is permitted to be moved (i.e., slid) along the cables 32 to adjust a location of the clip on the lead set 15. When the clip 62 has been positioned at the desired location, the second top portion 68 can be pivoted from an open position (FIG. 7) to a closed position (FIG. 8A) to lock the clip 62 in place on the lead set 15. The second top portion 68 can be toggled between the locked and unlock position as needed by a user. In one example implementation, the clip 62 comprises molded plastic. However, the clip 62 may comprise other materials (e.g., composites, metals) without departing from the scope of the disclosure.

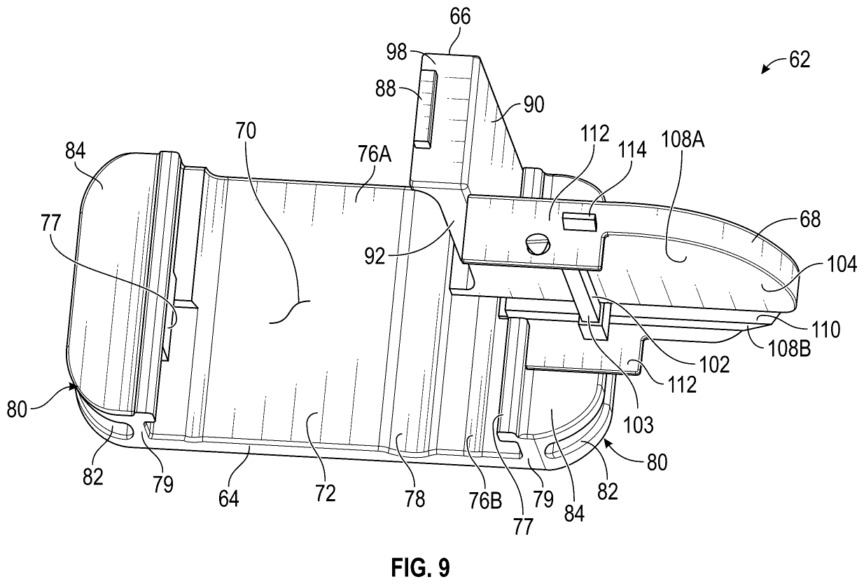

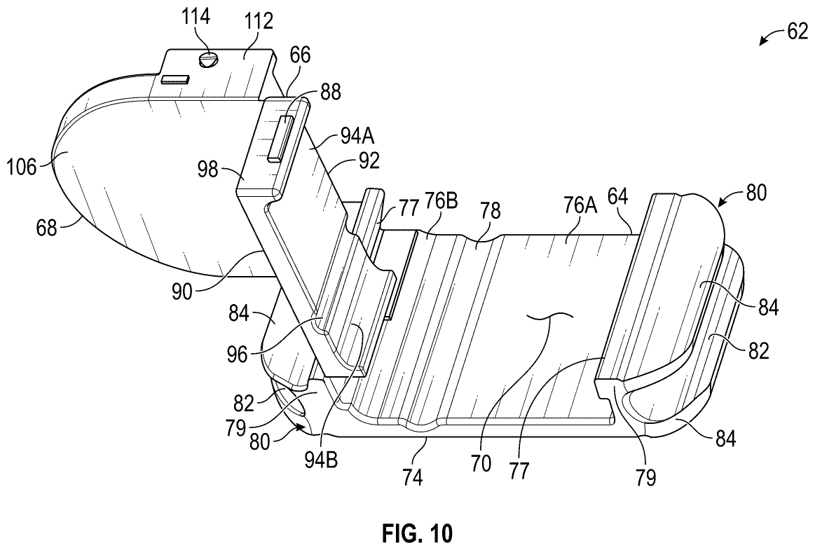

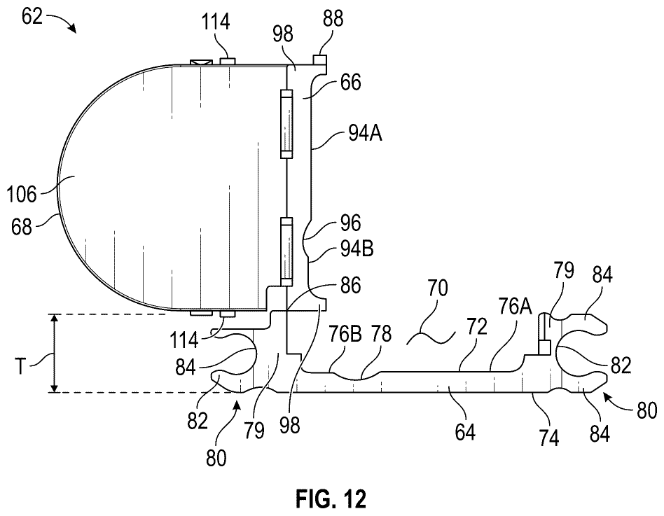

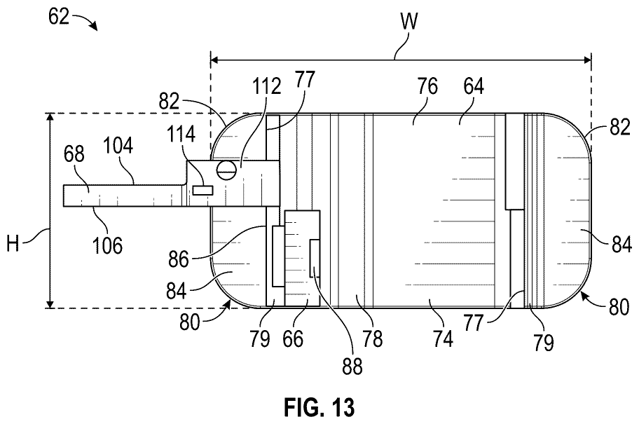

Referring to FIGS. 9-13, the base portion 64 may comprise a generally rectangular member defining an upper surface 72 and a bottom surface 74. A thickness T (FIG. 12) of the base portion 64 extends between the upper surface 72 and bottom surface 74. A height or length H of the base portion 64 extends between top and bottom edges of the rectangular member, and a width W of the base portion extends between opposite sides of the rectangular member (FIG. 13). The upper surface 72 defines planar (first) portions 76A, 76B and a recessed (second) portion 78 located between the planar portions. The recessed portion 78 extends along the height H of the base portion. The upper surface 72 provides a first engagement surface for engaging bottoms of the cables 32 of the lead set 15 when the wires are received in the space 70 of the clip 62. As shown in FIG. 6, the cables 32 extend along the height H of the clip 62, and as will be explained greater detail below, the planar portions 76A, 76B may be configured to support cables of a first size (i.e., diameter), and the recessed portion 78 may be configured to support a cable of a second size (i.e., diameter) greater than the first size. In the illustrated example, the first planar portion 76A may have a larger surface area than a second planar portion 76B, and only a single recessed portion 78 may be provided. For example, the first planar portion 76A may have a surface area that is at least twice the surface area of the second planar portion 76B. However, the upper surface 72 may have other configurations without departing from the scope of the disclosure. Additionally, the base portion 64 is configured such that the width W (FIG. 13) of the base portion is greater than the height H of the base portion. However, the size and shape of the base portion 64 could instead have any other shape and/or proportions. Moreover, the lower surface 72 and the upper surface 74 could have other shapes and/or dimensions without departing from the scope of the present disclosure. In general, the lower and upper surfaces 72, 74 may be shaped to conform to cables and/or tubes of different sizes. For example, the upper surface 72 and/or the recessed portion 78 may be altered in dimension(s) to either accommodate cable sets (or multiple cables to be managed) that include a larger number of individual cable that the seven cables described herein. For example, the upper surface 72 and thus the space 70 of the clip 62 may be widened or narrowed to accommodate a larger number of cables or a smaller number of cables, respectively. For example, the space 70 of the clip 62 may be widened or narrowed to accommodate anywhere from three (3) to twenty (25) cables. Further, the recessed portion 78 may be modified to accommodate a cable or tube with a larger diameter and/or smaller diameter than what is shown in the examples Figures. For example, the diameter of the recessed portion 78 may be reduced to accommodate a smaller diameter cable or may be increased to accommodate a larger diameter cable. Further, while only a single recessed portion 78 is shown in the figures, any number of recessed portions may be implemented without departing from the scope of this disclosure. In one example, the recessed portion 78 may be repositioned with respect to the upper surface 72 (e.g., may be located at a different location than shown in the Figures along the width of the upper surface 72). In another example, the recessed portion 78 may be omitted altogether if the clip 62 is intended to be used with cables that all have the same diameter or have substantially the same or similar diameter(s). Further, a distance between the upper surface 72 and planar portions 94A (FIG. 10) and/or 108A and/or a distance between planar portion 94A and bar 102 and/or slight recess 103 may be increased or decreased to modify the clip 62 for use with cables or sets of cables and/or tubes with larger and/or smaller diameters. For example, the distances noted above may be increased for use with cables and/or sets of cables with a larger diameter and/or may be decreased for use with cables and/or sets of cables with a smaller diameter.

The base portion 64 further comprises a pair of side walls 79 extending along the height H (FIG. 13) of the base portion 64. The pair of side walls may be substantially perpendicular to the upper surface 72. In particular, the side walls 79 extend upward from the upper surface 72. As will be explained in greater detail below, the first top portion 66 is pivotally attached to one of the side walls 79 of the base portion 64 so that upon pivoting the first top portion 66 to the closed position (e.g., as shown in FIG. 7), the space 70 between the base portion 64 and the first top portion is at least partially closed to slideably constrain one or more cables and/or tubes. Thus, a first side wall 79 of the base portion 64 defines the attachment surface for pivotally attaching the first top portion 66 to the base portion, and a second side wall 79 defines a securement component for securing the first top portion in the closed position. For example, the second side wall 79 may define a lip 77 or other engagement feature for retaining a catch 88 or other engagement feature on the first top portion 66. The first side wall 79 may also define a lip 77 for retaining a catch 114 or other engagement feature on the second top portion 68 as will be explained in greater detail below. However, it will be understood that other securement components having other configurations can be utilized without departing from the scope of the disclosure. For example, the aforementioned lip(s) and catch(s) may be swapped or reversed from the configuration described. Further the aforementioned engagement features may be configured to provide tactile feedback to a user when for example the lip 77 or other engagement features is engaged with the catch 88 and/or 114 or other engagement feature. For example, when enough force is provided by a user pivoting the first top portion 66 down and/or the second top portion 68 down toward the base portion 64, the catch 88 and/or 114 may “snap” or “click” into the lip 77 or vice versa to indicate to a user that the catch 88 and/or 114 is fully engaged with the lip 77 and the clip 62 is slidably constrained on the cables and/or tubes within the space 70. In various embodiments, the catch 88 can be configured so that once the base portion 64 and first top portion 66 are in the closed configuration (i.e., FIG. 7) via the catch 88, the clip may not be reopened (i.e., FIG. 6) to prevent the clip from disengaging from the cables 32. In the aforementioned example, the first top portion 66 may permanently engage with base portion 64 or otherwise be prevented from re-opening. In some examples, a specialized tool or other opening member may be required to re-open the first top portion 66 once engaged with the base portion 64.

Referring to FIGS. 9-13, a pair of optional cable/wire retainers 80 may be disposed on opposite sides of the base portion 64. The wire retainers 80 are configured to releasably retain cables 32 of the lead set 15. Thus, any cables 32 not retained in the space 70 of the clip 62 can be received in one of the wire retainers 80 (FIG. 8A) and constrained therein. Each wire retainer 80 may comprises a channel member 82 with a semi-circular or round inner surface extending along the height H of the base portion. The channel member 82 is defined by a pair of flanges 84 extending laterally outward from the side walls 79 of the base portion 64. The channel member 82 is configured to receive a wire 32 of the lead set 15 along a retainer axis RA (FIG. 8A) extending parallel to the height H of the base portion 64 (FIG. 8A). The flanges 84 may be resiliently deformable to receive one or more cables 32 and/or tubes into the channel 82 and to retain the wires in the channel. In one example, the inner diameter of the wire retainers 80 may be the same dimension or slightly smaller than the diameter of the cables 32 and/or tubes to be installed therein, thus providing a friction fit that either reduces or prevents sliding of the clip 62 with respect to the cables 32 and/or tubes. In another example, the inner diameter of the wire retainers 80 may be the same dimensions or slightly larger than the diameter of the cables 32 and/or tubes to be installed therein, thus providing some friction between the inner surfaces of the wire retainers 80 and the outer surface of the cables 32, which may allow the clip 62 to slide with respect to the cables 32 while still constraining the wire retainers 80 and the clip 62 to the cables 32. The base portion 64 and first and second top portions 66, 68 are configured to receive each of the cables 32 of the lead set 15 along a base axis BA (FIG. 8A) also extending parallel to the height H of the base portion 64. In one aspect of the disclosure, the base axis BA and retainer axis RA are coplanar. In the illustrated aspect of the disclosure, the channel member 82 comprises a C-shaped or rounded internal surface channel extending along the entire height H of the base portion 64. Thus, the channel member 82 is configured to generally match an outer surface of a cable 32 and/or tube received in the channel member 82. However, the channel member 82 could have different configurations without departing from the scope of the disclosure. For example, the channel member 82 could have other shapes and/or could extend only partly along the height H of the base portion 64. Additionally, the illustrated example shows two wire retainers 80. However, a single wire retainer 80 or more than two wire retainers may be provided without departing from the scope of the disclosure. In various embodiments, the clip 62 can be implemented without the cable/wire retainers 80 (e.g., where all of the cables in a use case may fit inside the base portion 64 of the clip 62). FIG. 8B shows an example of a lead set assembly 60A that may share features with or may be analogous with the lead set assembly 60 described herein but with one or more of the wire retainers 80 removed. In the aforementioned example, the cables 32 may instead all be contained within the clip 62 when the first top portion 66 is pivoted into engagement with the base portion 64.

Referring to FIGS. 7-13, the first top portion 66 is pivotally attached to the base portion 64 by a living hinge 86 or similar hinge feature. An axis of the hinge 86 extends along the height H of the base portion 64. As such, the first top portion 66 can be pivoted away from the base portion 64 to the open position for receiving the cables 32 and/or tubes and/or combinations thereof, and toward the base portion to the closed position to engage the catch 88 on the first top portion with the lip 77 on the base portion to secure the first top portion to the base portion. In the illustrated example, a single hinge 86 is provided between the first top portion 66 and the base portion 64. However, additional hinges (which may be living hinges as shown in the example described herein) may be provided. Additionally, an alternative hinge or connection mechanism could be used to connect the first top portion 66 to the base portion 64 without departing from the scope of the disclosure.

The first top portion 66 may comprise a generally rectangular member defining an upper surface 90 (FIGS. 9 and 10) and a bottom surface 92. The bottom surface 92 may define planar (first) portions 94A, 94B and a recessed (second) portion 96 located between the planar portions. The bottom surface 92 may provide a second engagement surface for engaging tops of the cables 32 and/or tube(s) of the lead set 15 when the wires are received in the space 70 of the clip 62. The planar portions 94A, 94B of the first top portion 66 may be configured for supporting wires of a first size (e.g., diameter), and the recessed portion 96 may be configured for supporting a wire of a second size (e.g., diameter) greater than the first size. In the illustrated example, a first planar portion 94A has a larger surface area than a second planar portion 94B, and a single recessed portion 96 is provided. For example, the first planar portion 94A may have a surface area that is at least twice the surface area of the second planar portion 94B. However, the bottom surface 92 could have other configurations without departing from the scope of the disclosure. For example, additional recessed portion(s) similar to recessed portion 96 may be included. In another example, recessed portion 96 may be omitted and the planar portions 94A and 94B may form a continuous surface. Additionally, the first top portion 66 is configured such that a height of the first top portion is less than the height H (FIG. 13) of the base portion. Thus, the first top portion 66 may be configured to cover only a portion of the space 70 of the base portion 64 when the first top portion 66 is pivoted downward. Further, when the second top portion 68 is folded downward the first top portion 66 and second top portion 68 may cover the space 70 of the base portion base portion 64. However, the size and shape of the first top portion 66 and/or the second top portion 68 could be varied or otherwise altered without departing from the scope of the disclosure. The shapes of the surfaces 90, and/or bottom surface 92 may be different than described without departing from the scope of this disclosure. The surfaces 90 and/or, 92 may be shaped to receive cables 32 and/or tubes of only one size or may have different shapes than described for receiving wires of different sizes.

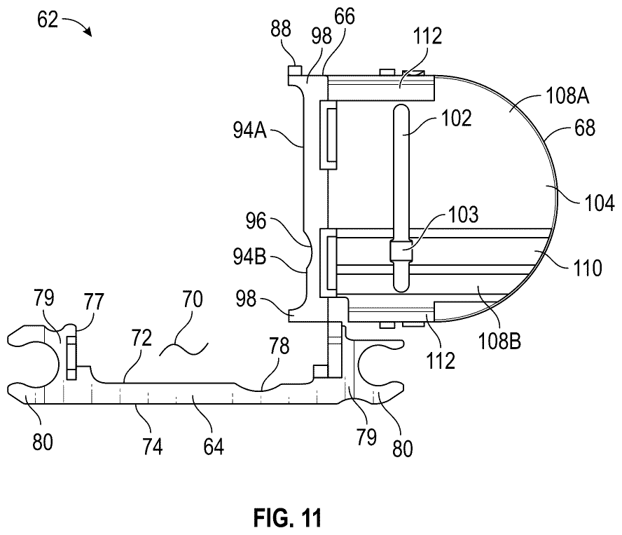

Turning to FIG. 11, the first top portion 66 may further comprises a pair of side walls 98 extending along the height of the first top portion and generally perpendicular from the bottom surface 92 (FIG. 10). In particular, the side walls 98 extend downward from the bottom surface 92. As previously explained, the first top portion 66 may be pivotally attached to one of the side walls 79 of the base portion 64 so that upon pivoting the first top portion 66 to the closed position, the space 70 between the base portion 64 and the first top portion is fully or partially closed. Thus, a first side wall 98 of the first top portion 66 may define the attachment surface for the hinge 86 to pivotally attach or otherwise engage the first top portion 66 to the base portion 64, and a second side wall 98 of the first top portion includes the catch 88 or other engagement feature for securing the first top portion 66 in the closed position when the catch 88 engages with or hooks under a lip 77 or other engagement feature of the base portion 64. However, it will be understood that other securement components having other configurations can be utilized without departing from the scope of the disclosure. For example, the aforementioned lip(s) and catch(s) may be swapped or reversed from the configuration described. Further the aforementioned engagement features may be configured to provide tactile feedback to a user when for example the lip 77 or other engagement features is engaged with the catch 88 or other engagement feature. For example, when enough force is provided by a user pivoting the first top portion 66 down toward the base portion 64, the catch 88 may “snap” or “click” into the lip 77 or vice versa to indicate to a user that the catch 88 is fully engaged with the lip 77 and the clip 62 is slidably constrained on the cables and/or tubes within the space 70.

Referring to FIGS. 7-13, the second top portion 68 is pivotally attached to a top edge of the first top portion 66 by a pair of hinges 100, which may be living hinges. An axis of the hinges 100 extends perpendicular to the height H (FIG. 13) of the base portion 64 and along the width W of the base portion 64. Thus, the hinges 100 extend along an axis that is orthogonal to the axis of the hinge 86. As such, after the first top portion 66 has been pivoted to the closed position (FIG. 7), the second top portion 68 can be pivoted away from the base portion 64 to the open position permitting the clip 62 to be slid along the cables 32, and toward the base portion to the closed position to engage a locking bar 102 (FIG. 9) or other engagement feature on a bottom surface 104 of the second top portion 68 with the cables 32 and/or tubes to lock the clip in place long the lead set 15, for example. In the illustrated example, a pair of hinges 100, which may comprise a pair of single living hinges, is provided between the second top portion 68 and the first top portion 66. However, a single living hinge or hinge or more than two living hinges or hinges may be provided. Additionally, an alternative hinge or connection mechanism could be used to connect the second top portion 68 to the first top portion 66 without departing from the scope of the disclosure.

In the illustrated embodiments, the second top portion 68 comprises a generally semicircular member with an upper surface 106 and the bottom surface 104. However, the second top portion 68 can be a variety of different geometric shapes without departing from the spirit of the disclosure (e.g., square or rectangle). The bottom surface 104 may define a planar (first) portions 108A and/or 108B (FIG. 11) and a recessed (second) portion 110 located between the planar portions 108A and/or 108B. The bottom surface 104 may provide a third engagement surface for engaging tops of the cables 32 of the lead set 15 when the wires are received in the space 70 of the clip 62. As described above for the first top portion 66, the planar portions 108A and/or 108B of the second top portion 68 may be configured for supporting cables and/or tubes of a first size (i.e., diameter), and the recessed portion 110 may be configured for supporting a cable or tube of a second size (i.e., diameter) greater than the first size. When the second top portion 68 is pivoted to the closed position, the planar portions 108A and/or 108B may be aligned with respective planar portions 94A and/or 94B on the first top portion 66, and the recessed portion 110 in the second top portion may be aligned with the recessed portion 96 in the first top portion to form a continuous recessed channel. This configuration provides a combined support surface that may extend along the entire height H of the base portion 64. In the illustrated example, a first planar portion 108A has a larger surface area than a second planar portion 108B, and a recessed portion 110 is provided. For example, the first planar portion 108A may have a surface area that is at least twice the surface area of the second planar portion 108B. However, the bottom surface 104 could have other varying or different configurations without departing from the scope of the disclosure. For example, additional recessed portion(s) similar to recessed portion 110 may be included. In another example, recessed portion 110 may be omitted and the planar portions 108A and/or 108B may form a continuous surface.

Additionally, the second top portion 68 may be configured such that a height of the second top portion 68, when pivoted to the closed position, is sufficient to cover the section of the base portion 64 not covered by the pivoted-closed or otherwise engaged first top portion 66. Thus, when pivoted closed, the second top portion 68 is configured to cover a top section (alternatively referred to as a second portion) of the base portion 64 while the first top portion 66 covers the bottom section (alternatively referred to as a first portion) of the base portion. However, the size and shape of the second top portion 68 could be varied and the examples shown and described are not intended to be limiting. In some examples, the extended or semi-circular shape of the second top portion 68 may provide additional leverage and/or a larger gripping surface, which may make it easier for a user to pivot the second top portion 68 into a closed position, and/or to move the second top portion 68 to an open position. The shown extended portion may make it significantly easier for a user to move the second top portion 68 to an open position when cables and/or tubes are constrained in the clip 62. As noted above, the semi-circular shape of the top portion 68 is merely provided as an example. Any alternative shape or configuration that functions as a lever or otherwise makes it easier for a user to open and/or close the second top portion 68 may be implemented.

Additionally, the planar portions 94A, 94B, 108A, and/or 108B of the first and second top portions 66, 68 may oppose the planar portions 76A, 76B of the base portion 64, and the recessed portions 96, 110 of the first and second top portions may oppose the recessed portion 78 of the base portion with the top portions are pivoted to their closed positions. Therefore, the planar portions and recessed portion provide channels that extend through the space 70 for supporting the cables 32 and/or tubes. The shapes of the bottom surface 106 and the top surface 104 may be other than described herein within the scope of the present invention.

The second top portion 68 further comprises a pair of side walls 112 extending along the height of the second top portion and generally perpendicular from the bottom surface 104. In particular, the side walls 112 extend downward from the bottom surface 104. As previously explained, the second top portion 68 may be pivotally attached to a top of the first top portion 66 so that upon pivoting the second top portion 68 to the closed position, the first top portion 66 and second top portions 68 are locked place on lead set 15 and/or any cables/tubes in the space 70. In particular, in the above example, the top surface 106 of the second top portion 68 becomes aligned (i.e., coplanar) with the top surface 90 of the first top portion 66, and catches or other engagement features 114 on each of the side walls 112 engage respective lips or other engagement features 77 on the base portion 64 to lock the second top portion 68 in the closed position. Additionally, the bar 102 on the bottom surface 104 of the second top portion 68 applies a force (e.g., friction and/or compression force) to the tops of any cables 32 and/or tubes constrained between the bar 102 and upper surface 72 of the base portion 64 to provide a friction and/or compression engagement preventing sliding and/or the relative movement of the clip 62 along the lead set 15. The bar 102 may extend across a width of the second top portion 86 and a slight recess 103 is provided in the bar. While the bar is shown with a rounded geometry, in other embodiments the bar can have a triangular geometry to more aggressively engage the cables 32 to even further reduce movement of the clip 62. The recess 103 in the bar 102 may be aligned with the recess 110 in the bottom surface 104 of the second top portion 86 to accommodate the larger size wire as described above. However, it will be understood that other securement components having other configurations can be utilized without departing from the scope of the disclosure. For example, as noted herein the aforementioned lip(s) and catch(s) may be swapped or reversed from the configuration described. Further the aforementioned engagement features may be configured to provide tactile feedback to a user when for example the lip 77 or other engagement features is engaged with the catch 114 or other engagement feature. For example, when enough force is provided by a user pivoting the first top portion 66 down toward the base portion 64, the catch 114 may “snap” or “click” into the lip 77 or vice versa to indicate to a user that the catch 114 is fully engaged with the lip 77 and the clip 62 is slidably constrained on the cables and/or tubes within the space 70.

When it is desirable to adjust the position of the clip 62 and/or remove the clip from the lead set 15, the second top portion 68 can be pivoted back to the open position to unlocking the clip from the lead set 15. At this point, the clip 62 can be slid along the cables 32 of the lead set 15 to adjust the location of the clip along the cables and/or tubes. Alternatively, if removal of the clip 62 is desired, the first top portion 66 can then be pivoted from the closed position to the open position to open up the space 70 and allow the clip to be fully removed from the cables 32.

As described herein, the clip 62 has the unique configuration whereby the clip includes three (3) movably connected components that provide for a first configuration where the cables 32 and/or tubes of the lead set 15 are receivable within the space 70 of the clip, a second configuration where the clip is closeable around the cables and/or tubes to secure the clip to the wires while permitting the clip to be moved (e.g., slid) along the cables and/or tubes, and a third configuration where the clip is lockable in place along the cables and/or tubes of the lead set. More specifically, the clip 62 comprises a first pivotable connection between the base portion 64 and the first top portion 66, and a second pivotable connection between the first top portion and the second top portion 68. The first pivotable connection configures the clip 62 for movement between the first and second configurations, and the second pivotable connection configures the clip for movement between the second and third configurations.

Additionally, while arranging the clip 62 in the third configuration fixedly secures the clip around and to the cables and/or tubes 32, the clip still provides for releasable connection of separate cables and/or tubes via the wire retainers 80. Therefore, the clip 62 is configured to simultaneously provide both a fixed or permanent wire retention and a releasable wire retention for the cables and/or tubes 32 of the lead set 15. Moreover, by locating the wire retainers 80 on the sides of the base portion 64, the wires held in the wire retainers are positioned in substantially the same plane as the cables 32 held in the space 70 between the base portion and the first and second top portions 66, 68. This provides the clip 62 with a low profile thereby making the lead set assembly 60 with the clip 62 installed thereon less intrusive. In one example implementation, the wire retainers 80 do not extend below the base portion 64 or above the first and second top portions 66, 68 creating the low profile configuration of the clip 62.

In view of the above, it will be seen that the several objects of the invention are achieved and other advantageous results attained.

As various changes could be made in the above constructions without departing from the scope of the invention, it is intended that all matter contained in the above description and shown in the accompanying drawings shall be interpreted as illustrative and not in a limiting sense.

Aspects of the disclosure are described in the following clauses:

Clause 1. A cable management clip for use with a cable set comprising a plurality of cables, the cable management clip comprising: a base defining a first engagement surface for supporting the cable set; a cover movably attached to the base and defining a second engagement surface for contacting the plurality of cables of the cable set, the cover being movable between an open position permitting the plurality of cables of the cable set to be received in a space between the base and the cover, and a closed position where the cover at least partially closes the space between the base and the cover; and a locking tab movably attached to the cover and defining a third engagement surface for contacting the plurality of cables of the cable set, the locking tab being movable between an open position permitting the cable management clip to be moveably constrained along the plurality of cables of the cable set, and a closed position fixing the cable management clip in place along the plurality of cables of the cable set. In one aspect, usable with any of the aspects above, the cover pivots about a first axis and the locking tab pivots about a second axis that is nonparallel with respect to the first axis.

Clause 2. The cable management clip of any of the preceding clauses, wherein the cover is pivotally attached to the base by a first living hinge, and the locking tab is pivotally attached to the cover by a second living hinge.

Clause 3. The cable management clip of any of the preceding clauses, wherein the first living hinge has a pivot axis that extends perpendicularly to a pivot axis of the second living hinge.

Clause 4. The cable management clip any of the preceding clauses, wherein the base, cover, and locking tab are formed as one piece of material.

Clause 5. The cable management clip any of the preceding clauses, wherein the first engagement surface comprises a first portion and a second portion, the first portion being configured to engage at least one of the plurality of cables of a first size, and the second portion being configured to engage at least one of the plurality of cables of a second size larger than the first size.

Clause 6. The cable management clip of any of the preceding clauses, wherein the second engagement surface comprises a first portion and a second portion, the first portion of the second engagement surface being configured to engage cables of a first size, and the second portion of the second engagement surface being configured to engage cables of a second size larger than the first size.

Clause 7. The cable management clip any of the preceding clauses, wherein the third engagement surface comprises a first portion and a second portion, the first portion of the third engagement surface being configured to engage cables of a first size, and the second portion of the third engagement surface being configured to engage cables of a second size larger than the first size.

Clause 8. The cable management clip any of the preceding clauses, wherein the locking tab includes a locking bar on the third engagement surface, the locking bar being configured to apply a friction force to the plurality of cables of the cable set to fix the cable management clip in place along the plurality of cables when the locking tab is moved to the closed position.

Clause 9. The cable management clip any of the preceding clauses, wherein the base includes a first securement element and the cover include a second securement element that engages the first securement element when the cover is moved to the closed position to hold the cover in the closed position.

Clause 10. The cable management any of the preceding clauses, wherein the locking tab includes a third securement element that engages the first securement element when the locking tab is moved to the closed position to hold the locking tab in the closed position.

Clause 11. The cable management clip any of the preceding clauses, wherein the locking tab includes a fourth securement element and the base includes a fifth securement element, the fourth securement element engaging the fifth securement element when the locking tab is moved to the closed position to hold the locking tab in the closed position.

Clause 12. The cable management clip any of the preceding clauses, wherein the cable set comprises first cables, the cable management clip further comprising a cable retainer disposed on the base and configured for releasably retaining a second cable of the cable set to the cable management clip when the locking tab is in the closed position.

Clause 13. The cable management clip of any of the preceding clauses, wherein the cable retainer comprises a first cable retainer, the cable management clip further comprising a second cable retainer disposed on the base and configured for releasably retaining a third cable of the cable set to the clip when the locking tab is in the closed position.

Clause 14. The cable management clip of any of the preceding clauses in combination with the cable set.

Clause 15. The cable management clip of any of the preceding clauses further comprising an engagement feature that engages when the cover is moved to closed position, wherein the cover cannot be re-opened to the open position once the engagement features is engaged.

Clause 16. A cable management clip for use with a lead set comprising: a base defining a first engagement surface for supporting first cables of the lead set; a cover movably attached to the base and defining a second engagement surface for contacting the first cables of the lead set, the cover being movable between an open position permitting the first cables of the lead set to be received in a space between the base and the cover, and a closed position where the cover at least partially closes the space between the base and the cover; and a cable retainer disposed on the base and configured for releasably retaining a second cable of the lead set to the cable management clip. In some aspects, the cover pivots about a first axis and the locking tab pivots about a second axis that is nonparallel with respect to the first axis.

Clause 17. The cable management any of the preceding clauses, wherein the base includes a height extending between a top edge and a bottom edge of the base, and a width extending between opposite sides of the base, the first cables of the lead set extending along the height of the base, and a cable extension plane extending across the width of the base, the cable retainer being disposed on one of the sides of the base.

Clause 18. The cable management any of the preceding clauses, wherein the base and cover are configured to retain one of the first cables of the lead set along a first cable axis, and the cable retainer is configured to retain the second cable along a second cable axis, the first cable axis and second cable axis being disposed in the cable extension plane.

Clause 19. The cable management clip any of the preceding clauses, the cable retainer comprises a first cable retainer, the cable management clip further comprising a second cable retainer disposed on the base and configured for releasably retaining a third cable to the cable management clip when the cover is in the closed position, the second cable retainer being disposed on the other of the sides of the base.

Clause 20. The cable management clip of any of the preceding clauses, wherein the second cable retainer is configured to retain the third cable along a third cable axis, the first cable axis and third cable axis being disposed in the cable extension plane.

Clause 21. The cable management any of the preceding clauses, further comprising a locking tab movably attached to the cover and defining a third engagement surface for contacting the first cables of the lead set, the locking tab being movable to fix the cable management clip in place along the first cables, the cable retainer releasably retaining the second cable of the lead set to the cable management clip when the cable management clip is fixed in place along the first cables.

Clause 22. The cable management clip of any of the preceding clauses further comprising an engagement feature that engages when the cover is moved to closed position, wherein the cover cannot be re-opened to the open position once the engagement features is engaged

What is claimed is:

1. A cable management clip for use with a cable set comprising a plurality of cables, the cable management clip comprising:
a base defining a first engagement surface for supporting the cable set; a cover movably attached to the base and defining a second engagement surface for contacting the plurality of cables of the cable set, the cover being movable between an open position permitting the plurality of cables of the cable set to be received in a space between the base and the cover, and a closed position where the cover at least partially closes the space between the base and the cover; and a locking tab movably attached to the cover and defining a third engagement surface for contacting the plurality of cables of the cable set, the locking tab being movable between an open position permitting the cable management clip to be moveably constrained along the plurality of cables of the cable set, and a closed position fixing the cable management clip in place along the plurality of cables of the cable set, wherein the cover pivots about a first axis and the locking tab pivots about a second axis that is nonparallel with respect to the first axis.

2. The cable management clip of claim 1, wherein the cover is pivotally attached to the base by a first living hinge, and the locking tab is pivotally attached to the cover by a second living hinge.

3. The cable management clip of claim 2, wherein the first living hinge has a pivot axis that extends perpendicularly to a pivot axis of the second living hinge.

4. The cable management clip of claim 1, wherein the base, cover, and locking tab are formed as one piece of material.

5. The cable management clip of claim 1, wherein the first engagement surface comprises a first portion and a second portion, the first portion being configured to engage at least one of the plurality of cables of a first size, and the second portion being configured to engage at least one of the plurality of cables of a second size larger than the first size.

6. The cable management clip of any of claim 1, wherein the second engagement surface comprises a first portion and a second portion, the first portion of the second engagement surface being configured to engage cables of a first size, and the second portion of the second engagement surface being configured to engage cables of a second size larger than the first size.

7. The cable management clip of claim 1, wherein the third engagement surface comprises a first portion and a second portion, the first portion of the third engagement surface being configured to engage cables of a first size, and the second portion of the third engagement surface being configured to engage cables of a second size larger than the first size.

8. The cable management clip of claim 1, wherein the locking tab includes a locking bar on the third engagement surface, the locking bar being configured to apply a friction force to the plurality of cables of the cable set to fix the cable management clip in place along the plurality of cables when the locking tab is moved to the closed position.

9. The cable management clip of claim 1 wherein the base includes a first securement element and the cover include a second securement element that engages the first securement element when the cover is moved to the closed position to hold the cover in the closed position.

10. The cable management clip of claim 9, wherein the locking tab includes a third securement element that engages the first securement element when the locking tab is moved to the closed position to hold the locking tab in the closed position.

11. The cable management clip of claim 1, wherein the locking tab includes a fourth securement element and the base includes a fifth securement element, the fourth securement element engaging the fifth securement element when the locking tab is moved to the closed position to hold the locking tab in the closed position.

12. The cable management clip of claim 1, wherein the cable set comprises first cables, the cable management clip further comprising a cable retainer disposed on the base and configured for releasably retaining a second cable of the cable set to the cable management clip when the locking tab is in the closed position.

13. The cable management clip of claim 12, wherein the cable retainer comprises a first cable retainer, the cable management clip further comprising a second cable retainer disposed on the base and configured for releasably retaining a third cable of the cable set to the clip when the locking tab is in the closed position.

14. The cable management clip of claim 1 in combination with the cable set.

15. The cable management clip of claim 1, further comprising an engagement feature that engages when the cover is moved to closed position, wherein the cover cannot be re-opened to the open position once the engagement features is engaged.

16. A cable management clip for use with a lead set comprising: a base defining a first engagement surface for supporting first cables of the lead set; a cover movably attached to the base and defining a second engagement surface for contacting the first cables of the lead set, the cover being movable between an open position permitting the first cables of the lead set to be received in a space between the base and the cover, and a closed position where the cover at least partially closes the space between the base and the cover; and a cable retainer disposed on the base and configured for releasably retaining a second cable of the lead set to the cable management clip.

17. The cable management clip of claim 16, wherein the base includes a height extending between a top edge and a bottom edge of the base, and a width extending between opposite sides of the base, the first cables of the lead set extending along the height of the base, and a cable extension plane extending across the width of the base, the cable retainer being disposed on one of the sides of the base.

18. The cable management clip of claim 17, wherein the base and cover are configured to retain one of the first cables of the lead set along a first cable axis, and the cable retainer is configured to retain the second cable along a second cable axis, the first cable axis and second cable axis being disposed in the cable extension plane.

19. The cable management clip of claim 18, the cable retainer comprises a first cable retainer, the cable management clip further comprising a second cable retainer disposed on the base and configured for releasably retaining a third cable to the cable management clip when the cover is in the closed position, the second cable retainer being disposed on the other of the sides of the base.

20. The cable management clip of claim 19, wherein the second cable retainer is configured to retain the third cable along a third cable axis, the first cable axis and third cable axis being disposed in the cable extension plane.

21. The cable management clip of claim 16, further comprising a locking tab movably attached to the cover and defining a third engagement surface for contacting the first cables of the lead set, the locking tab being movable to fix the cable management clip in place along the first cables, the cable retainer releasably retaining the second cable of the lead set to the cable management clip when the cable management clip is fixed in place along the first cables, wherein the cover pivots about a first axis and the locking tab pivots about a second axis that is nonparallel with respect to the first axis.

22. The cable management clip of claim 16, further comprising an engagement feature that engages when the cover is moved to closed position, wherein the cover cannot be re-opened to the open position once the engagement features is engaged.
